(19) United States
(12) Patent Application Publication
(10) Pub. No.: US 2022/0326236 A1
(43) Pub. Date: Oct. 13, 2022

(54) METHODS FOR ASSESSING THE RISK OF DEVELOPING ACTIVE TUBERCULOSIS

(71) Applicant: Albert Einstein College of Medicine, Bronx, NY (US)

(72) Inventors: Jacqueline M. Achkar, Brooklyn, NY (US); Eustache Paramithiotis, Montreal (CA)

(73) Assignee: Albert Einstein College of Medicine, Bronx, NY (US)

(21) Appl. No.: 17/764,693

(22) PCT Filed: Oct. 2, 2020
(86) PCT No.: PCT/US20/54083

§ 371 Date: Mar. 29, 2022

Related U.S. Application Data
(60) Provisional application No. 62/909,696, filed on Oct. 2, 2019.

(51) Int. Cl. G01N 33/569; G01N 33/68

(52) U.S. Cl. CPC G01N 33/5695; G01N 33/6893; G01N 2800/56; G01N 2800/60

(57) ABSTRACT

This disclosure describes markers that are associated with active tuberculosis (TB) and demonstrates that the disclosed markers can be used as a biomarker for determining whether a subject has or is at risk of having active TB and for the early detection of HIV-associated TB. This disclosure also provides methods of screening subjects who are thought to be at risk for developing active TB, methods of determining the efficacy of therapeutic regimens for preventing or treating active TB, and methods of identifying anti-TB agents.

### STATEMENT REGARDING FEDERALLY SPONSORED RESEARCH

This invention was made with government support under AI117927 awarded by the National Institutes of Health. The government has certain rights in the invention.

### FIELD OF THE INVENTION

The present invention relates generally to methods for predicting the risk of developing active tuberculosis (TB) in a subject and more specifically to methods for predicting the risk of developing active TB (e.g., HIV-associated TB) in a subject based on determining a level of one or more biomarkers, such as nidogen-1 (NID-1).

### BACKGROUND OF THE INVENTION

Active tuberculosis (TB) is a major global public health problem and the leading cause of death among people living with HIV (PLHIV). To fill gaps in the current TB diagnostic armamentarium, accurate and easily detectable non-sputum-based biomarkers correlating with increasing M. tuberculosis (Mtb) infection activity are urgently needed. Such biomarkers are critical for predicting the risk of TB development, detecting the early onset of disease, and monitoring antituberculous treatment response. Furthermore, the need for such biomarkers is particularly high in the setting of HIV co-infection, a major risk factor for TB development (Denkinger C M, et al. J Infect Dis. 2015; 211 Suppl 2:S29-38). Although asymptomatic latent Mtb infection (LTBI) and TB are often seen as binary states, disease due to reactivation of endogenous remote infection is preceded by a continuum of increasing Mtb infection activity. Depending on the level of immunosuppression, this process can last months to years before developing symptomatic and clinically diagnosable TB. Similarly, TB due to exogenous new or reinfection is preceded by increasing infection activity, albeit at an often faster rate than that of more remote infection. Because HIV infection increases the risk for TB development 30-60 fold, the World Health Organization (WHO) recommends TB preventative drug therapy for all PLHIV living in TB endemic regions. However, the effect of preventative therapy is often not durable, and there are associated side-effects (Churchyard G J, et al. N Engl J Med. 2014; 370(4):301-10). Because only a portion of those infected with Mtb develop TB, easily detectable non-sputum-based biomarkers correlating with Mtb infection activity could serve as screening or triage tools to identify both PLHIV at risk for TB and those with early disease.

The only currently available simple (for use by first contact providers) point-of-care (POC) test for the rapid diagnosis of TB is based on the detection of the Mtb cell wall glycolipid lipoarabinomannan (LAM) in urine. Because this lateral flow test (AlereLAM) has an overall test sensitivity of below 50% for TB, which changes to around 56% in patients with advanced HIV, the World Health Organization (WHO) has endorsed its use only for HIV-infected patients with CD4 counts <100 cells/μl. A recently developed new generation urine lateral flow POC (FujiLAM) has shown enhanced sensitivity in PLHIV with a median CD4 cell count of 86 cells/ul (70% compared to 42% for the AlereLAM). Nevertheless, the detection of LAM in the urine of patients still relies on sufficient mycobacterial replication. Various other studies have investigated host TB biomarkers as indirect markers for Mtb infection. Many of these have limitations in study designs, restricting implications of the data for their clinical applicability (MacLean E, et al. Nat Microbiol. 2019; 4(5):748-58).

Accordingly, there is a strong need in the art for novel TB biomarkers and methods for rapid TB diagnosis in a subject so that appropriate actions can be taken to reduce such risk and to limit the spread of the disease.

### SUMMARY OF THE INVENTION

This disclosure addresses the need mentioned above in a number of aspects. In one aspect, this disclosure provides a method for assessing the risk of developing active TB in a subject. The method comprises (i) obtaining a sample from the subject; (ii) determining a level of one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof in the sample; (iii) comparing the determined level to a control level of the one or more markers; and (iv) determining the risk of developing active TB in the subject based on the difference between the determined level of the one or more markers and the control level of the one or more markers. In some embodiments, the one or more markers comprise NID-1.

In another aspect, this disclosure also provides a method of reducing the risk of developing active TB. The method comprises (a) assessing the risk of developing active TB in the subject by a method as described above; (b) selecting a therapeutic agent for the subject based on the determined risk of developing active TB; and (c) administering to the subject an effective amount of the therapeutic agent to modulate a level or an activity of the one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof, thereby reducing the risk of developing active TB in the subject. In some embodiments, the therapeutic agent is selected from the group consisting of: isoniazid, rifampin, rifapentine, rifabutin, pyrazinamide, ethambutol, streptomycin, kanamycin, amikacin, moxifloxacin, gatifloxacin, levofloxacin, ofloxacin, ciprofloxacin aminocinomerizone, capre, thiacetazone, clarithromycin, amoxicillin-clavulanic acid, imipenem, meropenem, clofazimine, viomycin, terizidone, TMS-207, PA-824, OPC-7683, LL-3858, SQ-109, and combinations thereof.

In another aspect, this disclosure further provides a method of assessing the effectiveness of a treatment in a subject. The method comprises (i) determining the level of one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof, in a sample from the subject after at least a portion of the treatment has been administered to the subject; (ii) comparing the determined level of the one or more markers with a control level of the one or more markers obtained from the subject prior to the initiation of the treatment; and (iii) determining that the treatment is effective if the determined level of the one or more markers is decreased as compared to the control level.

In yet another aspect, this disclosure additionally provides a method of treating a subject having active TB. The method comprises administering to the subject an effective amount of a therapeutic agent that modulates a level of one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof. In some embodiments, the therapeutic agent is selected from the group consisting of: isoniazid, rifampin, rifapentine, rifabutin, pyrazinamide, ethambutol, streptomycin, kanamycin, amikacin, moxifloxacin, gatifloxacin, levofloxacin, ofloxacin, ciprofloxacin aminocinomerizone, capre, thiacetazone, clarithromycin, amoxicillin-clavulanic acid, imipenem, meropenem, clofazimine, viomycin, terizidone, TMS-207, PA-824, OPC-7683, LL-3858, SQ-109, and combinations thereof.

In some embodiments, the subject is a mammal (e.g., human). In some embodiments, the subject is HIV positive (HIV+) or HIV negative (−). In some embodiments, the subject was previously diagnosed as having latent TB.

In some embodiments, the control level of the one or more markers is a level of the one or more markers in a sample obtained from a control subject that does not have active TB. In some embodiments, the control level of the one or more markers is a level of the one or more markers in a sample obtained from a control subject having latent TB, a respiratory disease, or a combination thereof. In some embodiments, the control level of the one or more markers is a level of the one or more markers in a sample obtained from a healthy subject.

In some embodiments, the level of the one or more markers or the control level of the one or more markers comprises an RNA level, a protein expression level or an activity of the one or more markers.

In some embodiments, the protein expression level of the one or more markers is determined using immunoassay or mass spectrometry. In some embodiments, the immunoassay is an electrochemiluminescence assay, an enhanced chemiluminescence assay, an enzyme-linked immunosorbent assay (ELISA), or a lateral-flow assay (LFA). In some embodiments, the mass spectrometry is matrix-assisted laser desorption/time of flight (MALDI/TOF) mass spectrometry, liquid chromatography quadruple ion trap electrospray (LCQ-MS), or surface-enhanced laser desorption ionization/time of flight (SELDI/TOF) mass spectrometry.

In some embodiments, the RNA level of the one or more markers is determined by RT-PCR.

In some embodiments, the sample is a bodily fluid sample or a tissue sample. In some embodiments, the sample is selected from the group consisting of blood, serum, and plasma. In some embodiments, the sample is a blood sample.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A and 2B are Volcano plots of trend analysis showing the significance and annual change for the differential expression of host proteins over time identified within the SA (South Africa; FIG. 1A) and US (FIG. 1B) cohorts. The negative log₁₀-transformed p-value corresponds with the significance of the splined curve slope from 24 months prior to TB diagnosis until time closest to TB diagnosis. The indicated proteins are those meeting criteria of both remaining with a significant slope (from 24 months prior to time of TB diagnosis) after adjusting for a false discovery rate (FDR) of 10% and being significantly differentially expressed (p<0.01) in the incident TB cases at the time of TB diagnosis compared to the asymptomatic control cohort subjects (Interferon-Gamma Release Assay (IGRA) positive and negative combined for SA, and Mantoux tuberculin skin test (TST) positive and negative combined for the US cohort).

FIGS. 2A, 2B, 2C, 2D, and 2E are a set of diagrams showing temporal expression patterns for individual host proteins meeting criteria for markers of increasing Mtb infection activity in both SA (SA) and US cohorts. Graphs show the local polynomial regression (LOESS) fit. Criteria consist of (i) a statistically significant slope (from 24 months prior to time of TB diagnosis until time of TB diagnosis) after adjusting for a false discovery rate (FDR) of 10%, and (ii) being significantly differentially expressed (p<0.01) in the incident TB cases at the time of TB diagnosis compared to the asymptomatic control cohort subjects (IGRA positive and negative combined for SA, and TST positive and negative combined for the US cohort). A2GL: leucine-rich alpha-2-glycoprotein, NID-1: nidogen-1, SCTM1: secreted and transmembrane protein 1, A1AG1:alpha-1-acid glycoprotein 1.

FIGS. 3A, 3B, 3C, and 3D are a set of diagrams showing temporal expression patterns for individual host proteins meeting criteria for markers of increasing Mtb infection activity in either SA (FIGS. 3A and 3C) or US cohort (FIGS. 3B and 3D). Graphs show local polynomial regression (LOESS) fit. Criteria consist of (i) a statistically significant slope (from 24 months prior to time of TB diagnosis until time of TB diagnosis) after adjusting for a false discovery rate (FDR) of 10%, and (ii) being significantly differentially expressed (p<0.01) in the incident TB cases at the time of TB diagnosis compared to the asymptomatic control cohort subjects (IGRA positive and negative combined for SA, and TST positive and negative combined for the US cohort). PIGR: polymeric immunoglobulin receptor, PLSL: plastin-2, VWF: von Willebrand factor.

FIGS. 4A, 4B, 4C, and 4D are a set of diagrams showing ingenuity pathway analysis (IPA) of the most significant molecular and cellular functions for host proteins associated with Mtb infection activity in the SA cohort (FIGS. 4A and 4B) or the US cohort (FIGS. 4C and 4D). FIGS. 4A and 4C show the top cellular and molecular functions (p<0.01) associated with host proteins generated by IPA's Diseases and Functions analysis. P-values refer to the association between a given protein and function and were calculated using a Right Tailed Fisher's Exact test. FIGS. 4B and 4D show the detailed networks for the proteins within the most significantly associated functions (−log(p-value)>6).

FIGS. 5A and 5B are a set of diagrams showing discrimination of patients with incident TB from asymptomatic controls at various time intervals prior to TB diagnosis. The area under the receiver operating curve (AUC) is shown for the SA cohort (FIG. 5A) and the US cohort (FIG. 5B). Controls consist of IGRA positive and negative combined for the SA and TST positive and negative combined for the US cohort.

FIGS. 6A and 6B are a set of diagrams showing the longitudinal nidogen-1 (NID-1) values of the HIV-infected subjects. FIG. 6A shows the longitudinal NID-1 values of the South African Sinikithemba (SK) HIV-infected cohort subjects. FIG. 6B shows the longitudinal NID-1 values of the US WIHS HIV-infected cohort subjects. The data show that among South African subjects NID-1 levels were already significantly elevated up to 6 months prior to diagnosis of TB compared to control subjects who did not develop TB, while among the US subjects they were significantly elevated around time of TB diagnosis compared to controls who did not develop TB.

FIGS. 7A and 7B are a set of diagrams showing the NID-1 levels of the SK HIV-infected subjects. Both panels show that in controls who did not develop TB NID-1 levels did not change significantly over time, regardless of tuberculin skin test (TST) or interferon-gamma release assay IGRA results, while they continuously increased over time in the incident TB cases.

FIGS. 8A and 8B are the receiver operation characteristics (ROC) curves based on NID-1 concentrations of the HIV-infected subjects. FIG. 8A shows the ROC curve for the SK HIV-infected subjects. FIG. 8B shows the ROC curve for the WIHS HIV-infected subjects. The panels show that NID-1 can discriminate patients with TB (at time of TB diagnosis) from asymptomatic TST/IGRA positive controls with (FIG. 8A) an AUC of 0.91 for the South African SK cohort subjects, and (FIG. 8B) an AUC of 0.8 for the US WHIS cohort subjects.

FIGS. 9A and 9B are a set of diagrams showing the NID-1 levels of the subject with or without previous TB history (FIG. 9A) and the plasma NID-1 concentrations at TB registration/diagnosis. The data show that patients with prior history of TB have significantly higher levels of NID-1 at time of TB diagnosis than subjects without prior history of TB.

### DETAILED DESCRIPTION OF THE INVENTION

This disclosure is based, at least in part, on the discovery of markers that are associated with active tuberculosis (TB). In particular, it shows that the level of the one or more markers were elevated in biological samples obtained from subjects having active TB or at risk of developing active TB, relative to control samples. This disclosure demonstrates that the disclosed markers can be used as a biomarker for determining whether a subject has, or is at risk of having, active TB and for the early detection of HIV-associated TB. In addition, this disclosure provides methods of screening subjects who are thought to be at risk for developing active TB, methods of determining the efficacy of therapeutic regimens for preventing or treating active TB, and methods of identifying anti-TB agents.

### A. Markers and Methods of Diagnosis and Treatment

a. Markers and Methods of Diagnosis

This disclosure is based upon the discovery of host proteins, such as NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, and S10A9, exhibiting increased expression levels that correlate with increasing Mtb infection activity. The level of any one marker or any combination of the markers as disclosed may be used in the methods of diagnosis or treatment, which are further described below.

Accordingly, in one aspect, this disclosure provides a novel method for identifying subjects having, or at risk of developing active TB (e.g., HIV-associated TB). In some embodiments, the method comprises (i) obtaining a sample from the subject; (ii) determining a level of one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof in the sample; (iii) comparing the determined level to a control level of the one or more markers; and (iv) determining the risk of developing active TB in the subject based on the difference between the determined level of the one or more markers and the control level of the one or more markers.

In some embodiments, the one or more markers may include any one or any combination of NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, and S10A9. In some embodiments, the one or more markers may include NID-1.

In some embodiments, the method comprises (i) obtaining a sample from the subject; (ii) determining a level of NID-1 in the sample; (iii) comparing the determined level to a control level of NID-1; and (iv) determining the risk of developing active TB in the subject based on the difference between the determined level of NID-1 and the control level.

In another aspect, this disclosure also provides a method of reducing the risk of developing active TB. The method comprises (a) assessing the risk of developing active TB in the subject by a method as described above; (b) selecting a therapeutic agent for the subject based on the determined risk of developing active TB; and (c) administering to the subject an effective amount of the therapeutic agent to modulate a level or an activity of the one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof, thereby reducing the risk of developing active TB in the subject.

The methods can also be used to determine whether a subject is suitable to be administered with an agent, i.e., a therapeutic agent (e.g., an agonist, antagonist, peptidomimetic, protein, peptide, nucleic acid, small molecule, or other drug candidate) to treat active TB. Alternatively, the methods can be used to selecting a treatment regimen suitable for a subject based on the risk level of developing active TB. Accordingly, In some embodiments, the method comprises (a) assessing the risk of developing active TB in the subject by a method as described above; (b) selecting a therapeutic agent for the subject based on the determined risk of developing active TB; and (c) administering to the subject an effective amount of the therapeutic agent to modulate a level or an activity of NID-1, thereby reducing the risk of developing active TB in the subject.

In some embodiments, the therapeutic agent is selected from the group consisting of: isoniazid, rifampin, rifapentine, rifabutin, pyrazinamide, ethambutol, streptomycin, kanamycin, amikacin, moxifloxacin, gatifloxacin, levofloxacin, ofloxacin, ciprofloxacin aminocinomerizone, capre, thiacetazone, clarithromycin, amoxicillin-clavulanic acid, imipenem, meropenem, clofazimine, viomycin, terizidone, TMS-207, PA-824, OPC-7683, LL-3858, SQ-109, and combinations thereof.

The disclosed methods can be practiced in conjunction with any other method(s) used by the skilled practitioner to diagnose, prognose, and/or monitor TB. For example, the methods of the invention may be performed in conjunction with any clinical measurement of TB known in the art, including PCR and nucleic amplification based tests, or detection of TB antigens in the urine such as urinary lipoarabinomannan. serological, cytological and/or detection (and quantification, if appropriate) of other molecular markers. In some embodiments, the methods are practiced in conjunction with an HIV test.

The term “determining” means methods that include detecting the presence or absence of marker(s) in the sample, quantifying the amount of marker(s) in the sample, and/or qualifying the type of biomarker. Measuring can be accomplished by methods known in the art and those further described herein.

As used herein, the various forms of the term “modulate” are intended to include stimulation (e.g., increasing or upregulating a particular response or activity) and inhibition (e.g., decreasing or downregulating a particular response or activity).

A “marker” or “biomarker” is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median level, e.g., expression level, of the biomarker in the different groups is calculated to be statistically significant. Common tests for statistical significance include, among others, t-test, ANOVA, Kruskal-Wallis, Wilcoxon, Mann-Whitney, and odds ratio. Biomarkers, alone or in combination, provide measures of relative risk that a subject belongs to one phenotypic status or another. As such, they are useful as markers for, e.g., disease (prognostics and diagnostics), therapeutic effectiveness of a drug (theranostics), and of drug toxicity.

A “level of a marker” or “the level of a biomarker” refers to an amount of a marker present in a sample being tested. A level of a marker may be either in absolute level or amount (e.g., μg/ml) or a relative level or amount (e.g., relative intensity of signals).

A “higher level” or an “increase in the level” of marker refers to a level of a marker in a test sample that is greater than the standard error of the assay employed to assess the level of the marker and can be at least twice, three, four, five, six, seven, eight, nine, or ten or more times the level of the marker in a control sample (e.g., a sample from a subject having latent TB, a subject having an “other respiratory disease” (ORD), an HIV− subject, an HIV+ subject, an HIV− subject having latent TB, and HIV+ subject having latent TB, an HIV− subject having an ORD, and HIV+ subject having an ORD, and/or, the average level of the marker in several control samples).

A “lower level” or a “decrease in the level” of a marker refers to a level of the marker in a test sample that is less than the standard error of the assay employed to assess the level of the marker and can be at least twice, three, four, five, six, seven, eight, nine, or ten or more times less than the level of the marker in a control sample (e.g., a sample from a subject having latent TB, a subject having an ORD, an HIV− subject, an HIV+ subject, an HIV− subject having latent TB, and HIV+ subject having latent TB, an HIV− subject having an ORD, and HIV+ subject having an ORD, and/or, the average level of the marker in several control samples).

The term “control level” refers to an accepted or pre-determined level of a marker, which is used to compare the level of the marker in a sample derived from a subject. In one embodiment, the control level of a marker is based on the level of the marker in a sample(s) from a subject(s) having latent TB. In another embodiment, the control level of a marker is based on the level of the marker in a sample(s) from a subject(s) having an ORD. In another embodiment, the control level of a marker is based on the level of the marker in a sample(s) from a subject(s) that is HIV− or HIV+. In another embodiment, the control level of a marker is based on the level of the marker in a sample(s) from a subject(s) that is HIV− subject and has latent TB. In another embodiment, the control level of a marker is based on the level of the marker in a sample(s) from a subject(s) that is HIV+ and has latent TB. In another embodiment, the control level of a marker is based on the level of the marker in a sample(s) from a subject(s) that is HIV− subject and has an ORD. In another embodiment, the control level of a marker is based on the level of the marker in a sample(s) from a subject(s) that is HIV+ subject and has an ORD, and/or, the average level of the marker in several control samples. In one embodiment, the control level of a marker in a sample from a subject is a level of the marker previously determined in a sample(s) from the subject. In yet another embodiment, the control level of a marker is based on the level of the marker in a sample from a subject(s) prior to the administration of a therapeutic agent or therapy for TB. In another embodiment, the control level of a marker is based on the level of the marker in a sample(s) from a subject(s) having active TB that is not contacted with an agent. In another embodiment, the control level of a marker is based on the level of the marker in a sample(s) from a subject(s) having latent TB that is not contacted with an agent. In another embodiment, the control level of a marker is based on the level of the marker in a sample(s) from a subject(s) having active TB that is contacted with an agent. In another embodiment, the control level of a marker is based on the level of the marker in a sample(s) from a subject(s) having latent TB that is contacted with an agent. In one embodiment, the control level of a marker is based on the expression level of the marker in a sample(s) from an animal model of TB, a cell, or a cell line derived from the animal model of TB.

Alternatively, population-average values for a “control” level of expression of a marker may be used. In other embodiments, the “control” level of a marker may be determined by determining the level of a marker in a subject sample obtained from a subject before the onset of active TB, from archived subject samples, and the like.

In some embodiments, the control level of the one or more markers is a level of the one or more markers in a sample obtained from a control subject that does not have active TB. In some embodiments, the control level of the one or more markers is a level of the one or more markers in a sample obtained from a control subject having latent TB, a respiratory disease, or a combination thereof. In some embodiments, the control level of the one or more markers is a level of the one or more markers in a sample obtained from a healthy subject.

The level of the one or more markers in a sample obtained from a subject may be determined by any of a wide variety of well-known techniques and methods, which transform a marker within the sample into a moiety that can be detected and quantified. Non-limiting examples of such methods include analyzing the sample using immunological methods for detection of proteins, protein purification methods, protein function or activity assays, nucleic acid hybridization methods, nucleic acid reverse transcription methods, and nucleic acid amplification methods, immunoblotting, Western blotting, Northern blotting, electron microscopy, mass spectrometry, e.g., MALDI-TOF and SELDI-TOF, immunoprecipitations, immunofluorescence, immunohistochemistry, enzyme-linked immunosorbent assays (ELISAs), e.g., amplified ELISA, quantitative blood-based assays, e.g., serum ELISA, quantitative urine-based assays, flow cytometry, Southern hybridizations, array analysis, and the like, and combinations or sub-combinations thereof.

For example, an mRNA sample may be obtained from the sample from the subject (e.g., blood, serum, bronchial lavage, mouth swab, biopsy, or peripheral blood mononuclear cells, by standard methods) and expression of mRNA(s) encoding a marker in the sample may be detected and/or determined using standard molecular biology techniques, such as PCR analysis. For example, a method of PCR analysis can be reverse transcriptase-polymerase chain reaction (RT-PCR). Other suitable systems for mRNA sample analysis include microarray analysis (e.g., using Affymetrix's microarray system or Illumina's BeadArray Technology).

It will be readily understood by the ordinarily skilled artisan that essentially any technical means established in the art for detecting the level of a marker at either the nucleic acid or protein level can be used to determine the level of a marker as disclosed herein.

In some embodiments, the level of a marker in a sample is determined by detecting a transcribed polynucleotide or portion thereof, e.g., mRNA, or cDNA, of a marker gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, RNase protection assays (Melton et al., (1984) Nuc. Acids Res. 12:7035-56), Northern blotting, in situ hybridization, and microarray analysis.

Other known methods for detecting a marker at the protein level include methods such as electrophoresis, capillary electrophoresis, high-performance liquid chromatography (HPLC), thin-layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, and Western blotting.

In some embodiments, the level of the one or more markers or the control level of the one or more markers comprises an RNA level, a protein expression level or an activity of the one or more markers. In some embodiments, a level of NID-1 or a control level of NID-1 comprises an RNA level or a protein expression level of NID-1. In some embodiments, the protein expression level of the one or more markers (e.g., NID-1) is determined using immunoassay or mass spectrometry. In some embodiments, the immunoassay is an electrochemiluminescence assay, an enhanced chemiluminescence assay, an enzyme-linked immunosorbent assay (ELISA), or a lateral-flow assay (LFA). In some embodiments, the mass spectrometry is matrix-assisted laser desorption/time of flight (MALDI/TOF) mass spectrometry, liquid chromatography quadruple ion trap electrospray (LCQ-MS), or surface-enhanced laser desorption ionization/time of flight (SELDI/TOF) mass spectrometry. In some embodiments, the RNA level of the one or more markers is determined by RT-PCR.

The level of NID-1 in a test sample can be evaluated by obtaining a test sample from a test subject and contacting the test sample with a compound or an agent capable of detecting the nucleic acid (e.g., RNA) or the protein or a fragment thereof. The expression level of NID-1 can be measured in a number of ways, including measuring the RNA encoded by the gene.

In some embodiments, NID-1 RNA or protein samples can be isolated from biological samples using any of the well-known procedures. For example, biological samples can be lysed in a guanidinium-based lysis buffer, optionally containing additional components to stabilize the NID-1 RNA or protein. In some embodiments, the lysis buffer can contain purified RNAs as controls to monitor the recovery and stability of RNA from cell cultures. Examples of such purified RNA templates include the Kanamycin Positive Control RNA from PROMEGA (Madison, Wis.), and 7.5 kb Poly(A)-Tailed RNA from LIFE TECHNOLOGIES (Rockville, Md.). Lysates may be used immediately or stored frozen at, e.g., −80° C. Optionally, total RNA can be purified from cell lysates (or other types of samples) using silica-based isolation in an automation-compatible, 96-well format, such as the RNEASY purification platform (QIAGEN, Inc., Valencia, Calif.). Other RNA isolation methods are contemplated, such as extraction with silica-coated beads or guanidinium. Further methods for RNA isolation and preparation can be devised by one skilled in the art. The present methods can also be performed using crude samples (e.g., blood, serum, plasma, or cell lysates), eliminating the need for isolating RNA or protein. RNAse inhibitors or protease inhibitors are optionally added to the crude samples. The level of RNA can be evaluated with nucleic acid amplification, e.g., by standard PCR (U.S. Pat. No. 4,683,202), RT-PCR (Bustin S. J Mol Endocrinol. 25:169-93, 2000), quantitative PCR (Ong Y. et al., Hematology. 7:59-67, 2002), real-time PCR (Ginzinger D. Exp Hematol. 30:503-12, 2002), and in situ PCR (Thaker V. Methods Mol Biol. 115:379-402, 1999), or any other nucleic acid amplification method, followed by the detection of the amplified molecules using techniques known in the art. In some embodiments, the RNA level of NID-1 can be determined by RT-PCR.

In some embodiments, the sample is a bodily fluid sample or a tissue sample. In some embodiments, the sample is selected from the group consisting of blood, serum, and plasma. In some embodiments, the sample is a blood sample.

The term “sample” as used herein refers to a collection of similar cells or tissue isolated from a subject, as well as tissues, cells, and fluids present within a subject. The term “sample” includes any body fluid (e.g., blood fluids, lymph, gynecological fluids, cystic fluid, urine, ocular fluids, and fluids collected by bronchial lavage and/or peritoneal rinsing), or a cell from a subject. In one embodiment, the tissue or cell is removed from the subject. In another embodiment, the tissue or cell is present within the subject. Other subject samples include teardrops, serum, cerebrospinal fluid, feces, sputum, and cell extracts.

The term “body fluid” or “bodily fluid” refers to any fluid from the body of an animal. Examples of body fluids include, but are not limited to, plasma, serum, blood, lymphatic fluid, cerebrospinal fluid, synovial fluid, urine, saliva, mucous, phlegm, and sputum. A body fluid sample may be collected by any suitable method. The body fluid sample may be used immediately or may be stored for later use. Any suitable storage method known in the art may be used to store the body fluid sample: for example, the sample may be frozen at about −20° C. to about −70° C. Suitable body fluids are acellular fluids. “Acellular” fluids include body fluid samples in which cells are absent or are present in such low amounts that the miRNA level determined reflects its level in the liquid portion of the sample, rather than in the cellular portion. Such acellular body fluids are generally produced by processing a cell-containing body fluid by, for example, centrifugation or filtration, to remove the cells. Typically, an acellular body fluid contains no intact cells; however, some may contain cell fragments or cellular debris. Examples of acellular fluids include plasma or serum, or body fluids from which cells have been removed.

In some embodiments, the subject is a mammal (e.g., human). In some embodiments, the subject is HIV positive (HIV+). In some embodiments, the subject was previously diagnosed as having latent TB.

As used herein, the terms “patient” or “subject” refer to human and non-human animals, e.g., veterinary patients. The term “non-human animal” includes all vertebrates, e.g., mammals and non-mammals, such as non-human primates, mice, rabbits, sheep, dog, cat, horse, cow, chickens, amphibians, and reptiles. In one embodiment, the subject is a human, e.g., a pediatric and adult human. In one embodiment, a subject is HIV negative (HIV−). In another embodiment, the subject is HIV positive (HIV+). In another embodiment, the HIV status of the subject is unknown.

“Tuberculosis” (“TB”) is a multisystemic disease with myriad presentations and manifestations and is the most common cause of infectious disease-related mortality worldwide. Mycobacterium tuberculosis, a tubercle bacillus, is the causative agent of TB. The lungs are the most common site for the development of TB (pulmonary TB), and about 85% of patients with TB present with pulmonary complaints. Nonetheless, “extrapulmonary TB,” e.g., “disseminated TB,” can occur as part of a primary or late, generalized infection. Extrapulmonary TB can affect bones and joints, bronchus, eye, intestines, larynx, peritoneum, meninges, pericardium, lymph node, organs of the male or female urinary and reproductive systems, skin, stomach, and/or urinary systems.

When a person is infected with M. tuberculosis, the infection can take one of a variety of paths, most of which do not lead to actual TB. The infection may be cleared by the host immune system or suppressed into an inactive form called “latent tuberculosis infection,” with resistant hosts controlling mycobacterial growth at distant foci before the development of an active disease.

A subject has “latent tuberculosis (“LTB”) (also referred to as “latent tuberculosis infection” (“LTBI”)) when the subject is infected with M. tuberculosis but does not have active tuberculosis disease. Subjects having latent tuberculosis are not infectious. The main risk is that approximately 10% of these patients (5% in the first two years after infection and 0.1% per year thereafter but higher risk if immunosuppressed) will go on to develop “active tuberculosis” (“active TB”) and spread the disease at a later stage of their life if, for example, there is an onset of a disease affecting the immune system (such as AIDS) or a disease whose treatment affects the immune system (e.g., chemotherapy in cancer or systemic steroids in asthma or Enbrel, Humira or Orencia in rheumatoid arthritis; malnutrition).

The symptoms of a subject having TB are similar to the symptoms of a subject having an “ORD,” such as pneumonia, and include, for example, cough (e.g., coughing that lasts three or more weeks, coughing up blood or sputum, chest pain, or pain with breathing or coughing), unintentional weight loss, fatigue, fever, night sweats, chills, and/or loss of appetite.

Methods to diagnose a subject as having active and/or latent TB are known in the art. The primary screening method for TB infection (active or latent) is the Mantoux tuberculin skin test (TST) with purified protein derivative (PPD). An in vitro blood test based on interferon-gamma release assay (IGRA) with antigens specific for M. tuberculosis can also be used to screen for latent TB infection. Chest X-rays and culturing of sputum samples may also be used.

A subject having latent TB usually has a skin test or blood test result indicating M. tuberculosis infection; has a normal chest x-ray and a negative sputum test; has TB bacteria in his/her body that are alive, but inactive; does not feel sick (e.g., does not have a cough and/or fever); and cannot spread TB bacteria to others. A subject having active TB usually has a positive skin test or tuberculosis blood test, may have an abnormal chest x-ray, or positive sputum smear or culture; has overt indications of illness (e.g., cough and/or fever), and can spread the disease to others.

Human immunodeficiency virus (HIV) is a lentivirus (e.g., slowly-replicating retrovirus) that causes acquired immunodeficiency syndrome (AIDS), an infectious disease in which progressive failure of the human immune system leads to life-threatening opportunistic infections and/or cancer.

HIV-1 testing can be performed by an enzyme-linked immunosorbent assay (ELISA) to detect antibodies to HIV-1. Subjects are considered “HIV-negative” (“HIV−”) if samples from the subject have a nonreactive result from the initial ELISA unless new exposure to an infected partner or partner of unknown HIV status has occurred. Subject samples with a reactive ELISA result are retested in duplicate. If the result of either duplicate test is reactive, the subject specimen is reported as repeatedly reactive and undergoes confirmatory testing with a more specific supplemental test (e.g., Western blot or an immunofluorescence assay (IFA)). Only subject samples that are repeatedly reactive by ELISA and positive by IFA or reactive by Western blot are considered “HIV-positive” (“HIV+”) and indicative of HIV infection. Specimens that are repeatedly ELISA-reactive occasionally provide an indeterminate Western blot result, which may be either an incomplete antibody response to HIV in an infected person or nonspecific reactions in an uninfected person. Although IFA can be used to confirm infection in these ambiguous cases, this assay is not widely used. In general, a second specimen is collected more than a month later and retested for persons with indeterminate Western blot results. Although much less commonly available, nucleic acid testing (e.g., viral RNA or proviral DNA amplification method) can also help diagnosis in certain situations. In addition, a few tested specimens might provide inconclusive results because of a low quantity specimen. In these situations, a second specimen is collected and tested for HIV infection.

b. Methods for Assessing the Effectiveness of a Treatment

In another aspect, this disclosure further provides a method of assessing the effectiveness of a treatment in a subject. The method comprises (i) determining the level of one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof in a sample from the subject after at least a portion of the treatment has been administered to the subject; (ii) comparing the determined level of the one or more markers with a control level of the one or more markers obtained from the subject prior to the initiation of the treatment; and (iii) determining that the treatment is effective if the determined level of the one or more markers is decreased as compared to the control level.

By way of example, the level of the one or more markers in a pair of samples (a first sample not subjected to the treatment regimen and a second sample subjected to at least a portion of the treatment regimen) is assessed. A modulation in the level of expression of the one or more markers in the first sample, relative to the second sample, is an indication that the therapy is effective for treating a subject having active TB and/or inhibiting the progression of TB to disseminated TB (or a complication associated with disseminated TB (e.g., spinal and kidney meningitis, peritonitis, pericarditis, bone and joint complications, fallopian tube infection, bowel infection, Adult respiratory distress syndrome (ARDS), liver inflammation, lung failure, and/or relapse of the disease) in a subject having TB.

By way of example, changes in expression levels and/or trends of one or more markers as disclosed, such as NID-1, in a biological sample, e.g., peripheral blood samples, can provide an early indication of recovery or lack thereof. For example, a further increase (or decline) or persistently-altered gene expression levels the one or more disclosed markers indicate a poor prognosis, i.e., lack of improvement. Accordingly, the disclosed markers can be used to assess the efficacy of a treatment for TB and outcomes of post-treatment recovery of TB.

Also provided by this disclosure is a method of monitoring a treatment for active TB in a subject. For this purpose, gene expression levels of one or more markers as disclosed, such as NID-1, can be determined for test samples from a subject before, during, or after undergoing a treatment. The magnitudes of the changes in the levels as compared to a baseline level are then assessed. A decrease in the magnitudes of the changes after the treatment indicates that the subject can be further treated by the same treatment. For example, a relative decrease in the expression level of the one or more markers is indicative of recovery from active TB or reduced risk of developing active TB. Conversely, further increase or persistent high expression levels of the one or more markers is indicative of lack of improvement.

c. Methods of Treatment

The subject having active TB may benefit from modulation of the expression and/or activity of the one or more markers (e.g., NID-1) as disclosed. Accordingly, this disclosure additionally provides a method of treating a subject having active TB and methods for reducing or inhibiting the development of complications associated with a disease in a subject. The method comprises administering to the subject an effective amount of a therapeutic agent (e.g., a modulator of the one or more markers) that modulates a level of one or more markers comprising NID-1.

The methods of “inhibiting” or “treating” include administration of a modulator of the disclosed marker(s) to a subject in order to cure or to prolong the health or survival of a subject beyond that expected in the absence of such treatment.

In some embodiments, the method may further comprise administering to the subject a second agent (e.g., therapeutic agent or therapy). For example, a modulator of the disclosed marker(s) can be administered in combination with (i.e., together with or linked to (i.e., an immunoconjugate)) cytotoxins, immunosuppressive agents, radiotoxic agents, and/or therapeutic antibodies. The second agent may include, but are not limited to, Isoniazid, Rifampin (Rifadin, Rimactane), Ethambutol (Myambutol), Pyrazinamide, streptomycin, vitamin D, Clarithromycin, Dapsone, Ofloxacin, Rifabutin, Non-nucleoside reverse transcriptase inhibitors (NNRTIs; e.g., efavirenz (Sustiva), etravirine (Intelence) and nevirapine (Viramune, Nucleoside reverse transcriptase inhibitors (NRTIs; e.g., Abacavir (Ziagen), and the combination drugs emtricitabine and tenofovir (Truvada), and lamivudine and zidovudine (Combivir), Protease inhibitors (PIs; e.g., atazanavir (Reyataz), darunavir (Prezista), fosamprenavir (Lexiva) and ritonavir (Norvir), Entry or fusion inhibitors, e.g., enfuvirtide (Fuzeon) and maraviroc (Selzentry), and Integrase inhibitors, e.g., Raltegravir (Isentress), or combinations thereof.

Modulators of the disclosed markers and the second agent or therapy can be administered in the same formulation or separately. In the case of separate administration, the modulators can be administered before, after or concurrently with the second agent or therapy. One agent may precede or follow administration of the other agent by intervals ranging from minutes to weeks. In embodiments where two or more different kinds of therapeutic agents are applied separately to a subject, one would generally ensure that a significant period of time did not expire between the time of each delivery, such that these different kinds of agents would still be able to exert an advantageously combined effect on the target tissues or cells.

The term “effective amount,” as used herein, refers to the amount of the modulators, which is sufficient to treat and/or inhibit the progression of active TB and/or a complication of TB in a subject when administered to a subject. An effective amount will vary depending upon the subject and the severity of the disease and age of the subject, the manner of administration, and the like, which can readily be determined by one of ordinary skill in the art. Dosage regimens may be adjusted to provide the optimum therapeutic response. An effective amount is also one in which any toxic or detrimental effects (i.e., side effects) of a modulator are minimized and/or outweighed by the beneficial effects.

The administration can be intravenous, intramuscular, intraperitoneal, or subcutaneous, or administered proximal to the site of the target. If desired, the effective daily dose of a modulator may be administered as two, three, four, five, six or more sub-doses administered separately at appropriate intervals throughout the day, optionally, in unit dosage forms. While it is possible for a modulator to be administered alone, the modulator can be administered as a pharmaceutical formulation (or composition).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the modulators may be administered once or twice weekly by subcutaneous injection or once or twice monthly by subcutaneous injection.

To administer a modulator used in the disclosed methods by certain routes of administration, it may be necessary to include the modulator in a formulation suitable for preventing its inactivation. For example, the modulator may be administered to a subject in an appropriate carrier, for example, liposomes or a diluent. Pharmaceutically acceptable diluents include saline and aqueous buffer solutions. Liposomes include water-in-oil-in-water CGF emulsions, as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active modulator, use thereof in pharmaceutical compositions is contemplated. Supplementary active compounds can also be incorporated with the modulator.

Modulators that can be used in the disclosed methods include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated and the particular mode of administration. The amount of active ingredient which can be combined with a carrier material to produce a single dosage form will generally be that amount of the modulator which produces a therapeutic effect. Generally, out of one hundred percent, this amount will range from about 0.001% to about 90% of active ingredient, e.g., from about 0.005% to about 70%, from about 0.01% to about 30%.

The phrases “parenteral administration” and “administered parenterally,” as used herein, means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns, therefore, are absent in the messenger RNA (mRNA) transcript. The mRNA functions during translation to specify the sequence or order of amino acids in a nascent polypeptide.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene products.” If the polynucleotide is derived from genomic DNA, expression may include splicing of the mRNA in a eukaryotic cell.

“Expression profile” refers to a genomic expression profile, e.g., an expression profile of microRNAs. Profiles may be generated by any convenient means for determining a level of a nucleic acid sequence e.g., quantitative hybridization of microRNA, cRNA, etc., quantitative PCR, ELISA for quantification, and the like, and allow the analysis of differential gene expression between two samples. A subject or patient sample, e.g., cells or a collection thereof, e.g., tissues, is assayed. Samples are collected by any convenient method known in the art. Nucleic acid sequences of interest are nucleic acid sequences that are found to be predictive, including the nucleic acid sequences of those described herein, where the expression profile may include expression data for 5, 10, 20, 25, 50, 100 or more of, including all of the listed nucleic acid sequences. The term “expression profile” may also mean measuring the abundance of the nucleic acid sequences in the measured samples.

As used herein, “treatment” or “treating,” or “palliating” or “ameliorating” are used interchangeably. These terms refer to an approach for obtaining beneficial or desired results, including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “prevent,” “preventing,” “prevention,” “prophylactic treatment” and the like refer to reducing the probability of developing a disorder or condition in a subject, who does not have, but is at risk of or susceptible to developing a disorder or condition.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “increased,” “increase” or “enhance” or “activate” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “decrease,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of the presence or absence of the disease or condition. Each factor or symptom that is considered to be indicative of the diagnosis of a particular disease does not need to be exclusively related to the particular disease; i.e., there may be differential diagnoses that can be inferred from a diagnostic factor or symptom. Likewise, there may be instances where a factor or symptom that is indicative of a particular disease is present in an individual that does not have the particular disease. The diagnostic methods may be used independently or in combination with other diagnosing and/or staging methods known in the medical art for a particular disease or disorder.

The term “prognosis” as used herein, refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy; instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average cure rate, a lower propensity for metastasis, a longer than expected life expectancy, and the like.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg, etc.) bodyweight,” even if the term “bodyweight” is not explicitly mentioned.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### B. EXAMPLES

### Example 1

This example describes the materials and methods used in the subsequent EXAMPLES below.

### Subjects and Study Design

Prospectively collected and stored samples from the adult SA and US HIV-infected cohort subjects up to two and a half years (30 months) before and one and a half years (18 months) post TB development were tested. In addition, the samples from HIV-infected cohort subjects who were asymptomatic and had not developed TB in a 1:2 case-control design were tested.

Cohort Descriptions.

Subjects from SA were enrolled within the Sinikithemba cohort which was based in Durban, and followed a few hundred HIV-infected men and women from 2003-2009 to study HIV clade C virus infection. Subjects from the US were enrolled within the Women's Interagency HIV Study (WIHS) cohort, one of the largest and longest US-wide cohorts. This cohort was established in 1993 to investigate the impact of HIV infection on women in the US and follows several thousand HIV-infected women. All subjects in both cohorts had documented HIV infection and were seen either around every 3 months (Sinikithemba) or every 6 months (WIHS). During follow-up visits, routine laboratory (e.g., CD4 counts and HIV viral load (VL)), physical exams and interviews were performed, and blood was drawn, aliquoted, and frozen at −80° C. until needed.

Ethics Statement.

Human blood sample collections for the performed studies were approved by the relevant institutional review and ethics boards, and all patients studied gave informed written consent.

Incident TB Case and LTBI Definitions.

Because subjects in both cohorts were followed for HIV progression and not specifically for TB, the time of TB diagnosis among Sinikithemba cohort subjects was considered equivalent to the date a physician recorded a diagnosis of TB in the medical records. For the WIHS subjects, it was the date a subject was reported to a US state department of health as having TB. Control subjects from the Sinikithemba cohort had results of a blood-based interferon-gamma release assay (IGRA; Elispot) available, and those from the WHIS cohort had Tuberculin skin test (TST) results available. As per guidelines, IGRA and TST positive subjects were considered Mtb infected (Lewinsohn D M, et al., Clin Infect Dis. 2017; 64(2):111-5). Among the asymptomatic controls, half were randomly selected with either a positive IGRA (SA Sinikithemba) or a positive TST (US WHIS). The other half were randomly selected among subjects with a negative IGRA or TST.

### Sample Processing and Data Acquisition

All plasma samples were aliquoted on the day of the blood draw and stored at −80° C. until testing. For the incident TB cases, consecutive longitudinal samples were processed. For each control subject, a single randomly-selected time-point was tested. The plasma samples from each cohort were processed and analyzed independently.

Randomization and Blinding.

All plasma samples were randomly coded by a computer program, and all personnel in testing were blinded as to the clinical information associated with each sample. Samples were grouped into blocks containing each of the clinical groups, and the order of the samples within each block was randomized. Each processing batch contained a balanced number of disease groups comprising around 21 test sera and 3 standard human serum quality control samples inserted at the beginning, middle, and end of each batch.

Sample Processing.

Samples were processed for LC-MS/MS as described (Achkar J M, et al. EBioMedicine. 2015; 2(9):1160-8; Bark C M, et al., EBioMedicine. 2017; 21:150-7). Briefly, samples were depleted of abundant proteins using an HSA/IgG column (Agilent Technologies, Mississauga, ON) in tandem with an IgY14 and Supermix (Sigma, Oakville, Ontario) column. The flow-through was digested with trypsin, freeze-dried, desalted, distributed into triplicate 96-well plates, and vacuum evaporated. Sample plates were stored at −20° C. until injection by LC-MS/MS.

Data Acquisition.

Acquisition of MRM-MS data was carried out as described (Achkar J M, et al. EBioMedicine. 2015; 2(9):1160-8; Bark C M, et al., EBioMedicine. 2017; 21:150-7). Briefly, peptide separation was achieved using a BioBasic C18 column (Thermo; 320 μm×150 mm, 5 μm particle size). The optimal two transitions per peptide were determined using selected reaction monitoring (SRM)-triggered MS/MS on a QTRAP 5500 instrument (AB Sciex). The two most intense fragment ions (b or y fragment ions only) in the MS/MS spectrum and its elution time were determined for each acquired peptide, and the collision energy (CE) was optimized for all of the chosen transitions. Sample processing variability was measured using aliquots of pooled plasma that were inserted at regular intervals among the study samples and taken through the entire analysis. For the SA cohort, the process quality control sample CV was 16.8%, and for the US cohort, the process quality control sample CV was 16.1%.

Expression analysis of MRM-MS data acquired from the verification samples was performed using R version 3.3.1, platform x86_64-pcmingw32/x64 (64-bit). An intensity threshold (IT) below which the measure is deemed less reliable was determined empirically and set to 10,000 pre-normalization. A detection rate (DR) was defined as the proportion of samples within a group with a raw intensity value greater or equal to the IT. Transitions with DR below 50% in both incident TB and asymptomatic groups were excluded from expression analysis. Differential intensity (DI) ratios were calculated in pairwise comparisons for each transition as the ratio of the average normalized intensities of each group.

Statistical Methods

Analysis of Temporal Trends for Each Protein.

Because it was expected that proteins correlating with Mtb infection activity would show changes in temporal patterns of the intensities, a linear spline model was fitted with the change-points at TB diagnosis. Data from incident cases ranging from 24.5 months before to 12.5 months after TB diagnosis and the randomly selected time-point for all controls were included in the separately for SA and US cohorts performed trend analyses. To account for within-person correlation, a random intercept was included (Bates D, et al., Journal of Statistical Software. 2014; 67(1):1-48). The following model was fitted for each protein:

yᵢⱼ=μ₀+zᵢβ₀+zᵢβ₁tⱼ+zᵢβ₂tⱼ1(tⱼ>0)+γᵢₖ+θᵢ+∈ᵢⱼ

where yᵢⱼ was the normalized protein intensity of i^{th} subject at j^{th} visit; tⱼ was the months before and after TB diagnosis; zᵢ was 1 if i^{th} subject was a case, 0 if control; μ₀ was the mean intensities among the controls who had the lowest level of CD4 at baseline; γᵢₖ (k=2, 3, 4) was the CD4 level effect for the second, third, and the highest level of CD4; θᵢ was the Gaussian random intercept for i^{th} subject; and εᵢⱼ was the Gaussian mean-zero residual with common variance.

For each protein, three hypotheses were tested: HO: 0=0, HO: 1=0, and HO: 2=0. The first test was the comparison between the controls and the cases at TB diagnosis. The second test examined whether the slope before TB diagnosis among the cases was flat. And, as a secondary objective, the third test examined whether the slope after TB diagnosis among the cases was different from the pre-diagnosis slope. It was controlled for type 1 error at 0.01 for the between-group (case-control) comparison, and for false discovery rate (FDR) at 10% for the pre-diagnosis trend test using Benjamini & Hochberg's method (Benjamini Y & Hochberg Y. Methodological, 1995; 57(1):289-300). Because the post-diagnosis test was a secondary objective, the results were not adjusted.

Prediction of TB.

Linear support vector machines (SVMs) (Cortes C, et al., Machine learning. 1995; 20(3):273-97; Chang C-C, et al., TIST, 2011; 2(3):1-27), a machine-learning method for binary outcomes, was recursively used to identify the proteins contributing the most to distinguishing cases from controls at 0-6 months, 6-12 months, 12-18 months, and 18-24 months prior to TB diagnosis. Rather than analyzing individual proteins, this method finds a set of proteins that work together in distinguishing two sample groups (Bachoo R M, et al., Proc Natl Acad Sci USA. 2004; 101(22):8384-9). To avoid overfitting and information leakage, both the variable selection and the model fitting steps were included in leave-one-out cross-validation. First, one test sample was randomly selected and fitted by SVM with remaining training samples with all 146 and 138 host proteins (for SA and US cohorts respectively) as predictors and calculated the probability that the test sample would be TB. Then, using backward variable selection, the protein with the least absolute linear coefficient at each iteration was removed until no proteins remained in the model. As each protein was removed, the TB probability of the test sample and which proteins remained in the model were recorded. And the process was repeated until each sample was once selected as a test sample. Thus, for each number of proteins between 1 and 146 or 138, the TB probability, true case-control status, and area under the curve (AUC) of ROC for all samples were calculated. The number of proteins (i.e., model complexity, k) that correspond with the highest AUC was chosen, and the k proteins that were most frequently selected in the model with that size k were selected. For this analysis, the intensities of proteins were scaled by their standard deviation across the samples (all cases and controls 30.5 months before and 12.5 months after TB diagnosis). These analyses were performed separately for the SA and US cohorts. All analyses were performed statistical software R (R Core Team (2013). R: A language and environment for statistical computing. R Foundation for Statistical Computing, Vienna, Austria).

### Example 2

Protein biomarkers predictive of onset of HIV-associated TB were identified using samples from the two distinct cohorts—the Sinikithemba cohort, representing SA as a region with high TB incidence and the WHIS cohort, representing the US as a region of low TB incidence. Within the Sinkithemba cohort, 31 subjects with incident TB (cases) were identified with one subject excluded due to lack of a sample within 6 months prior to TB diagnosis (Table 1). Within the WHIS cohort, 24 subjects with incident TB were identified (Table 2). Using a 1:2 case-control design, controls consisted of one half either IGRA+(SA) or TST+(US), and the other half IGRA or TST negative asymptomatic individuals.

TABLE 1
South African incident TB cases and controls

| | SA TB+ | | SA Non-TB | | |
|---|---|---|---|---|---|
| Characteristics | (n = 30) | | (n = 62) | | P-value |
| Female (%) | 22 | (73) | 47 | (76) | 0.80^{a} |
| Age, mean years | 37 | (12) | 35 | (9) | 0.30^{b} |
| (±SD) | | | | | |
| IGRA+ (%)^{c} | NA | | 31 | (50) | NA |
| CD4 cells/mm³, | 306 | (239-375) | 293 | (231-439) | 0.98^{e} |
| median (IQR)^{d} | | | | | |
| VL log median, | 5.03 | (4.32-5.35) | 4.29 | (3.42-4.79) | 0.002^{e} |
| copies/ml (IQR)^{d, f} | | | | | |

Subjects were all HIV+ and enrolled within the Sinikithemba cohort.
^{a}Chi-square test;
^{b}t test;
^{c}IGRA, Interferon-gamma release assay;
^{d}IQR, Interquartile range;
^{e}Mann-Whitney U test;
^{f}VL, Viral load.

TABLE 2
US incident TB cases and controls

| | US TB+ | | US Non-TB | | |
|---|---|---|---|---|---|
| Characteristics | (n = 24) | | (n = 48) | | P-value |
| Age, mean years | 39 | (7) | 38 | (6) | 0.71^{a} |
| (±SD) | | | | | |
| TST+ (%)^{b} | NA | | 24 | (50) | NA |
| CD4 cells/mm³, | 196 | (95-338) | 213 | (123-433) | 0.21^{d} |
| median (IQR)^{c} | | | | | |
| VL log median, | 4.49 | (3.74-5.51) | 3.79 | (2.77-4.93) | 0.004^{d} |
| copies/ml (IQR)^{c, e} | | | | | |

Subjects were all HIV+ and enrolled within the Women’s Interagency HIV Study (WIHS) cohort.
^{a}t test;
^{b}TST, Tuberculin skin test;
^{c}IQR, Interquartile range;
^{d}Mann-Whitney U test;
^{e}VL: Viral load.

For the SA cohort, 382 samples and, for the US cohort, 147 samples were analyzed. Protein biomarker data acquisition was carried out using a previously defined MRM-MS assay containing 163 proteins. These proteins have been previously shown to become differentially expressed in either US immigrants with TB compared to LTBI and other respiratory diseases stratified by HIV-infection status (Achkar J M, et al. EBioMedicine. 2015; 2(9):1160-8), or in newly Mtb-infected HIV negative Ugandan TB household contacts (Bark C M, et al., EBioMedicine. 2017; 21:150-7). For the SA samples, 147/163 (90%) and, for US samples, 138/163 (85%) of the targeted proteins were detected in greater than 50% of samples, the threshold for their inclusion in statistical analyses.

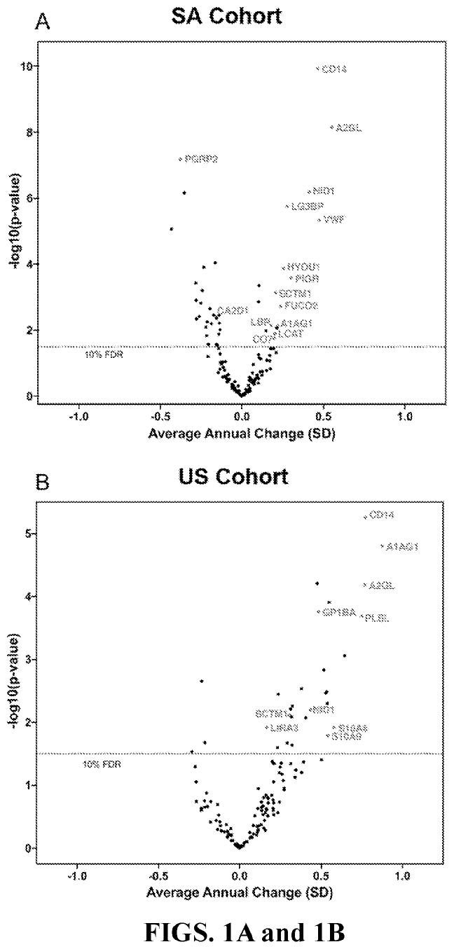

Both cohorts displayed significant differential expression changes in a subset of proteins leading up to TB diagnosis with many of these reversing significantly in the months post TB diagnosis, presumably due to treatment response. Proteins were considered to be markers of increasing Mtb infection activity if they met the criteria of both p<0.01 for the comparison of incident cases at the time of TB diagnosis to controls and p<0.05 for the trend analysis of protein intensity values within the incident TB cases (from 24.5 months prior to time of TB diagnosis). The magnitude of temporal protein expression changes (slope) for both SA and US cohort over the 24 months prior to TB diagnosis after adjusting for a false discovery rate (FDR) of 10% is depicted in FIGS. 1A and 1B. The SA cohort displayed a larger number of proteins meeting these criteria than the US cohort (n=15 versus 10, respectively) (Table 3).

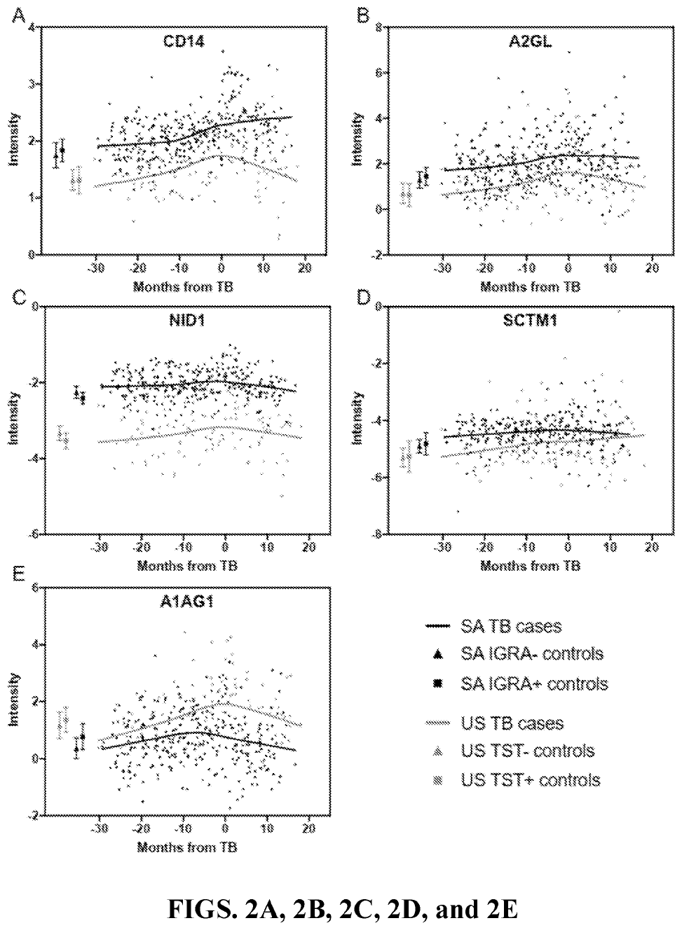

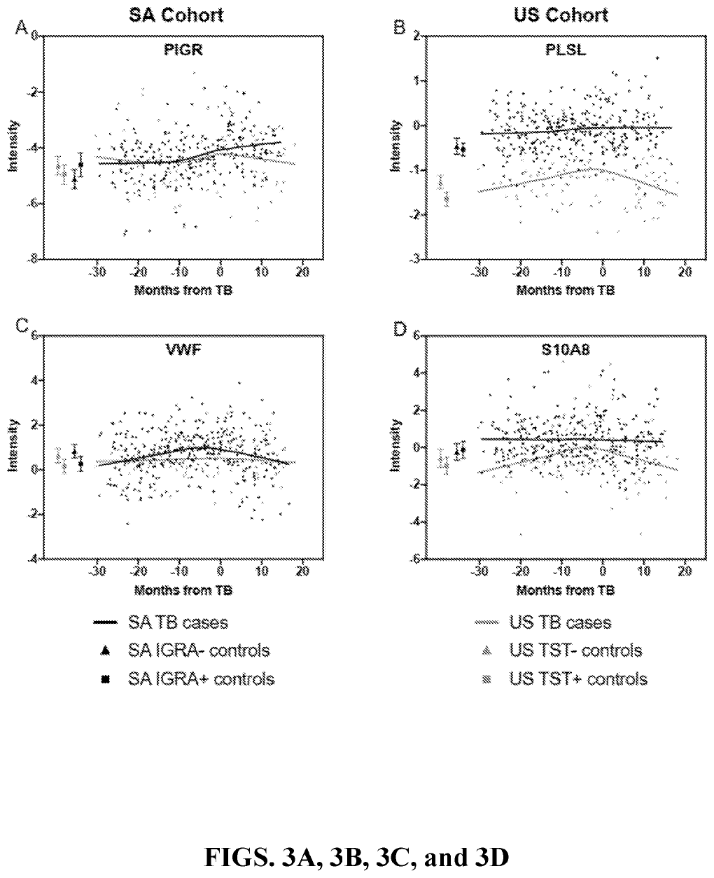

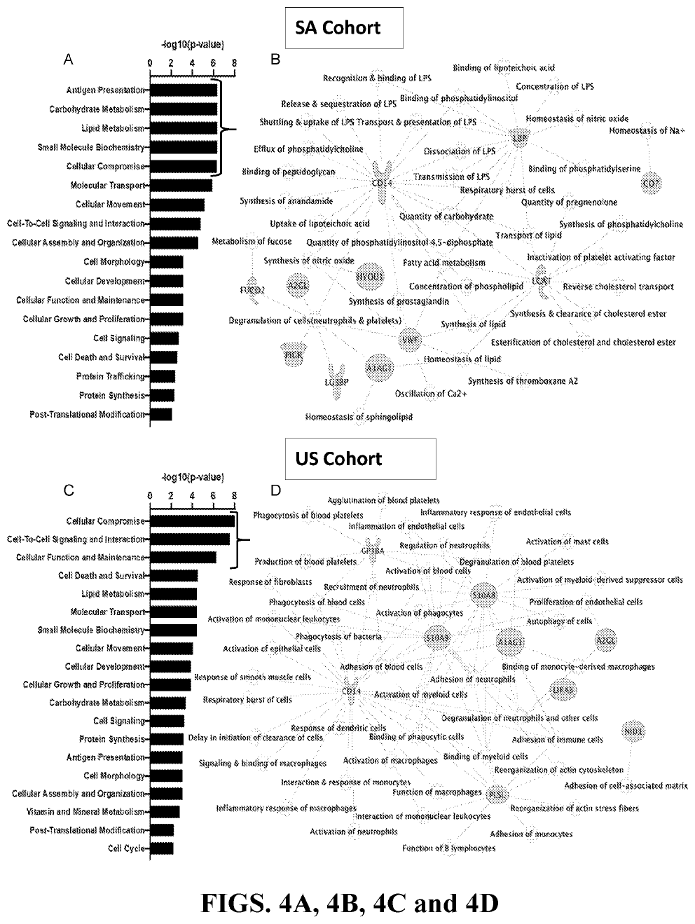

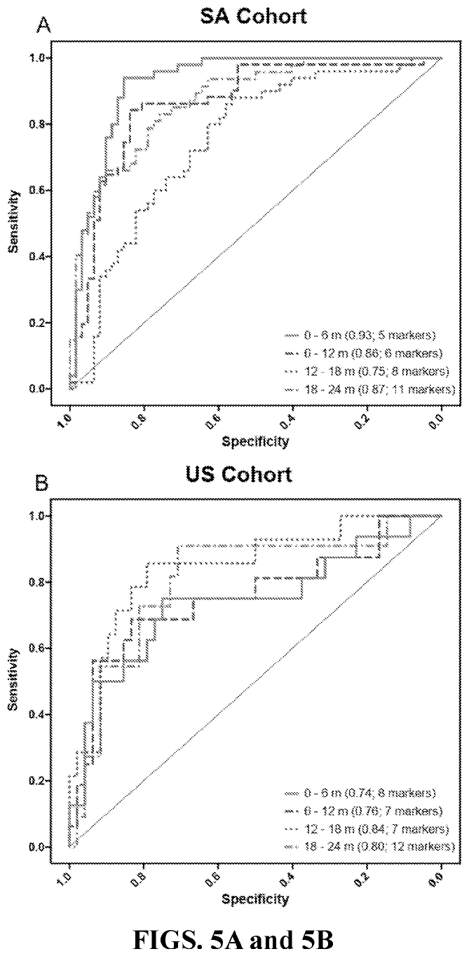

As observed in the TB antibody biomarker studies with biologically independent samples (Song L, et al., Mol Cell Proteomics, 2017; 16), the set of candidate biomarkers differed in part between SA and US incident TB cases. We identified 15 candidate proteins in the SA and 10 in the US cohort that met our selection criteria for increasing Mtb infection criteria—significance for both change over time after adjusting for FDR (10%) and significant difference (p<0.01) at time of TB diagnosis compared to asymptomatic cohort subjects who did not develop TB (FIGS. 1A-1B). Ingenuity pathway analysis suggests that these proteins have many significant molecular and cellular functions associated with Mtb infection activity (FIGS. 4A-4D). Some of them (e.g., NID-1, CD14, SCTM1) were also included in the panels of candidate markers predicting development of TB with high accuracy up to two years prior to TB diagnosis in our AUC analysis (FIGS. 5A-5B and Table 3). Most of the candidate proteins reversed significantly in the months following TB diagnosis, presumably due to treatment response (FIGS. 2A-2E and 3A-3D). Five markers, comprising 30% of the SA proteins and 50% of the US, overlapped between both cohorts. Specifically, these were soluble CD14, Alpha-2-glycoprotein (A2GL), Nidogen-1 (NID-1), secreted and transmembrane protein 1 (SCTM1), and Alpha-1-acid glycoprotein 1 (A1AG1)(FIGS. 1A-1B and 2A-2E). Applying a local polynomial regression (LOESS) fit (Cleveland W, et al., Local regression models. Statistical models in S; J M Chambers and T J Hastie eds.), 608 p. Wadsworth & Brooks/Cole, Pacific Grove, C A. 1992.), the temporal expression patterns for these proteins are shown in FIGS. 2A-2E. Additionally, the magnitude of differential expression patterns between the within-subject longitudinal analyses and the corresponding cross-sectional analyses differed in the SA subjects more than in the US subjects, indicating a higher and more variable Mtb infection activity in the SA subjects regardless of Mtb infection state.

FIGS. 3A, 3B, 3C, and 3D show examples of temporal expression patterns for individual host proteins meeting criteria for markers of increasing Mtb infection activity in either SA (FIGS. 3A and 3C) or US cohort (FIGS. 3B and 3D). Graphs show local polynomial regression (LOESS) fit. Criteria consist of (i) a statistically significant slope (from 24 months prior to time of TB diagnosis until time of TB diagnosis) after adjusting for a false discovery rate (FDR) of 10%, and (ii) being significantly differentially expressed (p<0.01) in the incident TB cases at time of TB diagnosis compared to the asymptomatic control cohort subjects (IGRA positive and negative combined for SA, and TST positive and negative combined for the US cohort). PIGR: polymeric immunoglobulin receptor, PLSL: plastin-2, VWF: von Willebrand factor.

FIGS. 4A, 4B, 4C, and 4D show ingenuity pathway analysis (IPA) of the most significant molecular and cellular functions for host proteins associated with Mtb infection activity in the SA cohort (FIGS. 4A and 4B) or the US cohort (FIGS. 4C and 4D). FIGS. 4A and 4C show the top cellular and molecular functions (p<0.01) associated with host proteins generated by IPA's Diseases and Functions analysis. P-values refer to the association between a given protein and function and were calculated using a Right Tailed Fisher's Exact test. FIGS. 4B and 4D show the detailed networks for the proteins within the most significantly associated functions (−log(p-value)>6).

FIGS. 5A and 5B show discrimination of patients with incident TB from asymptomatic controls at various time intervals up to two years prior to TB diagnosis. The area under the receiver operating curve (AUC) is shown for the SA cohort (FIG. 5A) and the US cohort (FIG. 5B). Controls consist of IGRA positive and negative combined for the SA, and TST positive and negative combined for the US cohort. Predictive candidates at the various time-intervals preceding TB diagnosis are listed in Tables 3 and 4.

TABLE 3
Composition of host protein biomarker panels distinguishing
incident TB cases prior to TB diagnosis from controls

| Time pre- | | | |
|---|---|---|---|
| TB | | SA cohort | US cohort |
| 0-6 | mo | CD248, HYOU1, NID-1, | CD14, CD166, DSG2, |
| | | PON1, VCAM1 | LAMP1, LRP1, NCAM2, |
| | | | R4RL2, VASN |
| 6-12 | mo | CD248, HYOU1, NID-1, | APOA1, APOA4, GP1BA, |
| | | PON1, VCAM1, APOA1 | LUM, NID-1, PLSL, SCTM1 |
| 12-18 | mo | APOA4, CBPN, CD14, | APOA4, CATA, COL11, |
| | | CPN2, LCAT, LUM, | LUM, MEGF8, PLSL, |
| | | LYAM1, PNPH | SCTM1 |
| 18-24 | mo | CD248, CSTN1, FUCO2, | AMPN, CA2D1, CBPQ, |
| | | HYOU1, MINP1, MMP2, | CNTN1, COL11, DPP4, |
| | | MYOC, NID-1, PON1, | HYOU1, ICAM1, LAMP1, |
| | | VASN, VCAM1 | LUM, PCOC1, PIGR |

HYOU1: hypoxia up-regulated protein 1,
NID-1: nidogen-1,
PON1: serum paraoxonase/arylesterase 1,
VCAM1: vascular cell adhesion protein 1,
DSG2: desmoglein-2,
LAMP1: lysosome-associated membrane glycoprotein 1,
LRP1: prolow-density lipoprotein receptor-related protein 1,
NCAM2: neural cell adhesion molecule 2,
R4RL2: reticulon-4 receptor-like 2,
VASN: vasorin,
APOA1: apolipoprotein A-I,
APOA4: apolipoprotein A-IV,
GP1BA: platelet glycoprotein Ib alpha chain,
LUM: lumican,
PLSL: plastin-2,
SCTM1: secreted and transmembrane protein 1,
CBPN: carboxypeptidase N catalytic chain,
CPN2: carboxypeptidase N subunit 2,
LCAT: lecithin-cholesterol acyltransferase,
LUM: lumican,
LYAM1: L-selectin,
PNPH: purine nucleoside phosphorylase,
CATA: catalase,
COL11: collectin-11,
MEGF8: multiple epidermal growth factor-like domains protein 8,
CSTN1: calsyntenin-1,
FUCO2: plasma alpha-L-fucosidase,
MINP1: multiple inositol polyphosphate phosphatase 1,
MMP2: matrix metalloproteinase-2,
MYOC: myocilin,
AMPN: aminopeptidase N,
CA2D1: voltage-dependent calcium channel subunit alpha-2/delta-1,
CBPQ: carboxypeptidase Q,
CNTN1: contactin-1,
DPP4: dipeptidyl peptidase 4,
ICAM1: intercellular adhesion molecule 1,
LAMP1: lysosome-associated membrane glycoprotein 1,
PCOC1: procollagen C-endopeptidase enhancer 1,
PIGR: polymeric immunoglobulin receptor

TABLE 4
Representative Genbank Accession Numbers for the markers

| | Genbank accession | |
|---|---|---|
| Protein | number | Gene name |
| NID1 | NM_002508 | NID1 |
| CD14 | NM_001174104 | CD14 |
| SCTM1 | NM_003004 | SECTM1 |
| A2GL | NM_052972 | LRG1 |
| A1AG1 | NM_000607 | ORM1 |
| PGRP2 | NM_001363546 | PGLYRP2 |
| LG3BP | NM_005567 | LGALS3BP |
| VWF | NM_000552 | VWF |
| HYOU1 | NM_006389 | HYOU1 |
| PIGR | NM_002644 | PIGR |
| FUCO2 | NM_032020 | FUCA2 |
| LBP | NM_004139 | LBP |
| LCAT | NM_000229 | LCAT |
| CO7 | NM_000587 | C7 |
| GP1BA | NM_000173 | GP1BA |
| PLSL | NM_002298 | LCP1 |
| LIRA3 | NM_006865 | LILRA3 |
| S10A8 | NM_001319196 | S100A8 |
| S10A9 | NM_002965 | S100A9 |

### DISCUSSION

Using plasma samples from prospectively followed HIV-infected subjects, host proteins that increase significantly from two years prior to until time of TB diagnosis were identified, indicating that these proteins correlate with increasing Mtb infection activity. Subjects from both cohorts, representing the US as a region of low TB incidence and SA as a region with high TB incidence, displayed significantly differential expression changes in partially overlapping subsets of proteins leading up to TB diagnosis. 15 candidate proteins in the SA and 10 in the US cohort were identified that met the selection criteria for increasing Mtb infection criteria—significance for both change over time after adjusting for FDR (10%) and significant difference (p<0.01) at time of TB diagnosis compared to asymptomatic cohort subjects who did not develop TB. Most of the candidate proteins reversed significantly in the months following TB diagnosis, presumably due to treatment response. Five proteins, CD14, A2GL, NID-1, SCTM1, and A1AG1, comprising 30% of the SA and 50% of the US candidates meeting the selection criteria for increasing Mtb infection activity, overlapped between both cohorts.

Although not in this combination, 4 of the 5 proteins overlapping in both cohorts have been identified in other, predominantly cross-sectional, TB biomarker studies, corroborating their relevance as markers of Mtb infection activity. CD14 is a pattern recognition receptor for microbial ligands and expressed on the surface of monocytes and polymorphonuclear cells. Its binding to bacterial and mycobacterial lipopolysaccharide and other cell wall components induces cellular activation and secretion of inflammatory cytokines. Upon activation, CD14 is shed in soluble form into serum.

A2GL (alpha-2-glycoprotein) belongs to the leucine-rich repeat group of proteins which are mainly involved in signal transduction and cell adhesion. Also referred to as LRG1 (leucine-rich glycoprotein 1), it has been shown to be increased in different inflammatory conditions, such as rheumatoid arthritis, inflammatory bowl disease, and acute appendicitis (Rainer T H, et al., Clin Biochem. 2017; 50(9):485-90; Serada S, et al., Annals of the rheumatic diseases. 2010; 69(4):770-4; Serada S, et al., Inflammatory bowel diseases. 2012; 18(11):2169-79). In humans, serum LRG1 levels in TB patients were significantly higher than those in healthy controls and declined one month post-treatment (Fujimoto, M., et al. Sci Rep 10(1): 3384.).

A1AG1 (alpha-1-acid glycoprotein 1, also referred to as orosomucoid1 (ORM1)) is an acute-phase protein which has immune-modulating effects including inhibition of leukocyte adhesion/migration, decreased neutrophil chemotaxis and superoxide production (Laine, E., et al. Inflammation 14(1): 1-9; Mestriner, F., et al., Proceedings Of The National Academy Of Sciences 104(49): 19595-19600; Spiller, F., et al. Diabetes 61(6): 1584-1591.). A1AG1 can be a potent inducer of the largely suppressive M2b macrophage subset leading to increased susceptibility to opportunistic infections. Extra-hepatic production of the protein in the lungs by alveolar macrophages and type 2 pneumocytes in experimental pulmonary TB in Balb/c mice suppressed cell mediated immunity and facilitated bacillary growth.

Also referred to as entactin, NID-1 is a multifunctional glycoprotein present in basement membranes, the sheet of extracellular matrix underlying epithelial and endothelial cells, and surrounding muscle, fat and neural tissues (Dedhar S, et al., J Biol Chem. 1992; 267(26):18908-14). NID-1 is involved in epithelial cell apopthosis, stimulates neutrophil adhesion and chemotaxis, and was recently further shown to regulate NK cell function by modulating cytotoxicity or interferon-gamma production (Gaggero S, et al., Oncoimmunology. 2018; 7(9):e1470730). Recently, it was identified by iTRAQ™-coupled LC-MS/MS among a subset of proteins elevated in tuberculous compared to malignant pleural effusions (Shi J, et al., Biomark Med. 2019; 13(2):123-33).

SCTM1 is a glycoprotein expressed by neutrophils, monocytes, and epithelial cells (Huyton T, et al., Biochimica Biophysica Acta. 2011; 1810(12):1294-301; Wang T, et al. J Leukoc Biol. 2012; 91(3):449-59). Its expression is inducible by INF-γ, leads to further INF-γ production, and, likely via C7-dependent mechanisms, SCTM1 costimulates CD4 and CD8 proliferation. It has been shown to modulate neutrophil and lymphocyte activation in airway epithelial cell infection with Streptococcus pneumoniae and human respiratory syncytial virus (Touzelet O, et al. Mol Cell Proteomics. 2020; 19(5):793-807; Kamata H, et al. American journal of respiratory cell and molecular biology. 2016; 55(3):407-18).

Among the molecular and cellular functions of the proteins correlating with increasing Mtb infection activity, cellular compromise was one of the most prominent in both cohorts (FIGS. 4A-4D). However, other top functional categories differed between the two cohorts, indicating potentially different interactions between Mtb and the host in PLHIV from SA versus those from the US. For example, antigen presentation, lipid, and carbohydrate metabolic functions were highly prominent in the protein set from SA, whereas cell-to-cell signaling and interactions, and cellular maintenance were more prominent in the US cohort. Given the likely higher exposure to Mtb in SA and the higher median CD4 counts in the SA compared to the US TB incident cases (˜300 vs. 200 cells/mm3), it is conceivable that Mtb exposure and/or the level of CD4 count could influence host-pathogen interactions.

### Example 3

People living with HIV (PLHIV) have an increased lifetime risk (30-60%) of developing active TB. Non-sputum-based biomarkers for both screening for and predicting the risk of HIV− associated TB development are urgently needed for early treatment initiation, especially in TB-endemic regions. Using mass spectrometry-based techniques, a novel immune-modulating extracellular matrix protein, NID-1, was identified among a subset of serum host proteins distinguishing active TB from other respiratory diseases in PLHIV.

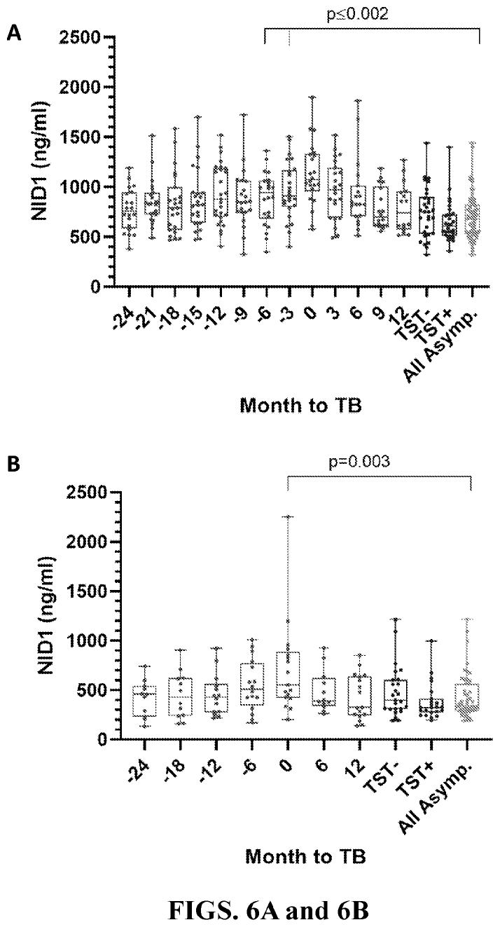

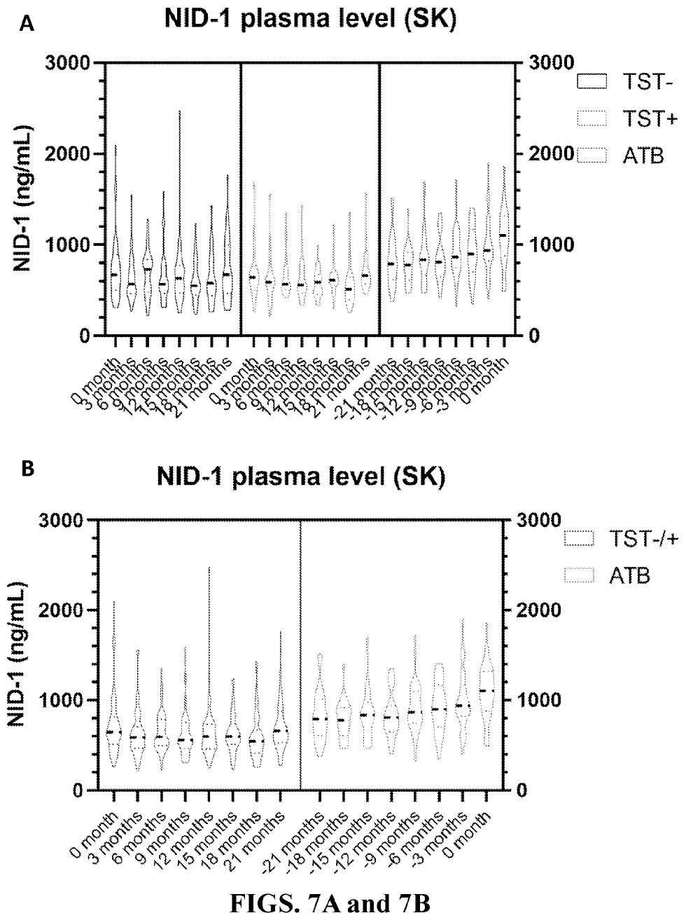

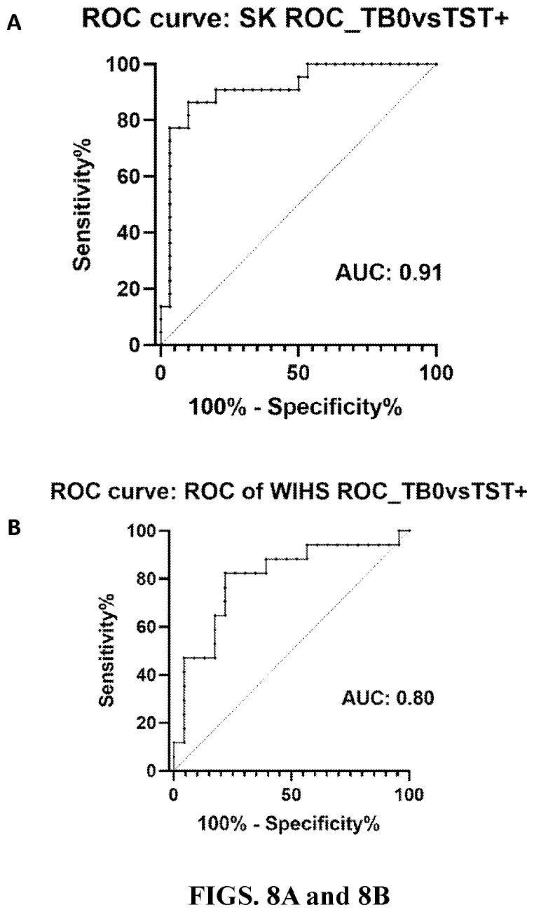

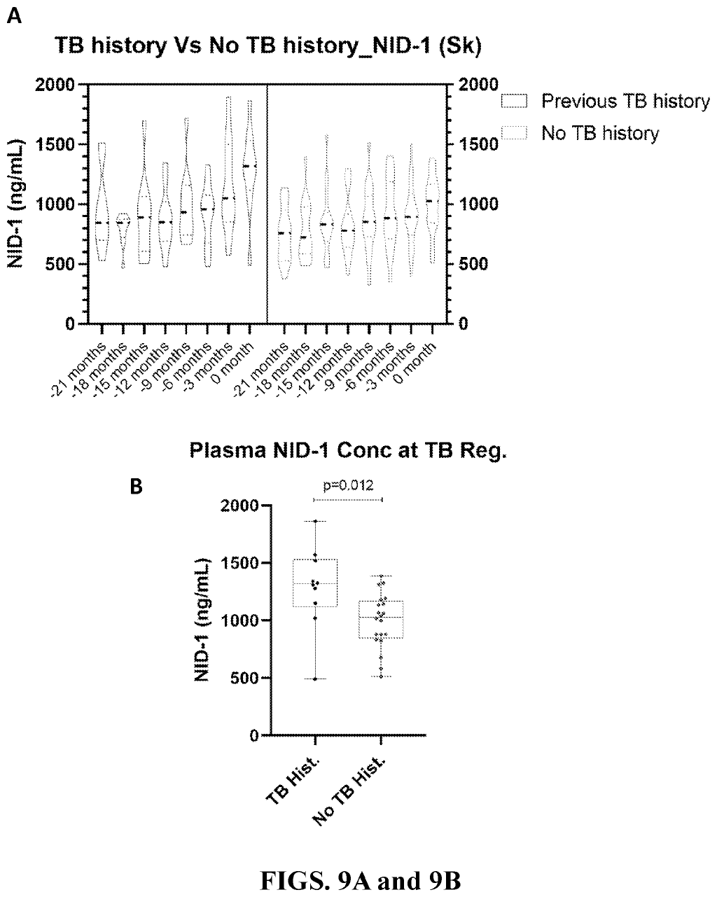

With plasma from longitudinal US and South African (SA) HIV+ cohorts, the expression of NID-1 in PLHIV who progressed to active TB compared to cohort subjects who did not develop TB was validated (FIGS. 6A-6B; FIGS. 7A-7B; FIGS. 8A-8B; and FIGS. 9A-9B). Using targeted sandwich ELISAs, plasma NID-1 levels in consecutive plasma samples ranging from 2 years before to a year post-TB diagnosis in US (n=24) and SA (n=30) HIV-positive incident TB cases and in US (n=47) and SA (n=61) HIV-infected controls (1:2 matched case-control) were measured. Half of the control subjects had signs of latent TB infection (LTBI) as indicated by either a positive Tuberculin skin test (TST; US) or interferon-gamma release assay (IGRA; Elispot; SA), and the other half had negative TST or IGRA. Because several SA incident TB subjects had a history of prior TB (n=10), the NID-1 levels at the time of TB diagnosis between patients and those without a history of prior TB were further compared.

The NID-1 levels did not differ significantly between TST positive and negative controls in the US, or IGRA positive and negative controls in the SA cohort. Compared to LTBI controls, NID-1 was significantly elevated in both the US and SA PLHIV 6 months before (p<0.05), and at the time of TB diagnosis (p<0.001). In patients with prior TB history, NID-1 levels at the time of TB diagnosis were significantly higher compared to those with a first-time TB diagnosis (p=0.012). AUCs for the detection of TB at the time of diagnosis were 0.80 in the US and 0.91 in the SA cohort compared to LTBI controls.

The data demonstrate that NID-1 can serve as a valuable non-sputum-based biomarker for the early detection of HIV-associated TB.

1. A method for assessing the risk of developing active TB in a subject, comprising:
obtaining a sample from the subject;
determining a level of one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof in the sample;
comparing the determined level to a control level of the one or more markers; and
determining the risk of developing active TB in the subject based on the difference between the determined level of the one or more markers and the control level of the one or more markers.

2. A method of reducing the risk of developing active TB, comprising:
assessing the risk of developing active TB in the subject by the method of claim 1;
selecting a therapeutic agent for the subject based on the determined risk of developing active TB; and
administering to the subject an effective amount of the therapeutic agent to modulate a level or an activity of the one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof, thereby reducing the risk of developing active TB in the subject.

3. A method of assessing the effectiveness of a treatment in a subject, comprising:
determining the level of one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof in a sample from the subject after at least a portion of the treatment has been administered to the subject;
comparing the determined level of the one or more markers with a control level of the one or more markers obtained from the subject prior to the initiation of the treatment; and
determining that the treatment is effective if the determined level of the one or more markers is decreased as compared to the control level.

4. A method of treating a subject having active TB, comprising administering to the subject an effective amount of a therapeutic agent that modulates a level of one or more markers selected from NID1, CD14, SCTM1, A2GL, A1AG1, PGRP2, LG3BP, VWF, HY0U1, PIGR, UC02, LBP, A1AG1, LCAT, CO7, PG1BA, PLSL, LIRA3, S10A8, S10A9, and combination thereof.

5. The method of claim 1, wherein the one or more markers comprise NID-1.

6. The method of claim 1, wherein the subject is HIV positive (HIV+).

7. The method of claim 1, wherein the subject is HIV negative (HIV−).

8. The method of claim 1, wherein the subject was previously diagnosed as having latent TB.

9. The method of claim 1, wherein the control level of the one or more markers is a level of the one or more markers in a sample obtained from a control subject that does not have active TB.

10. The method of claim 1, wherein the control level of the one or more markers is a level of the one or more markers in a sample obtained from a control subject having latent TB, a respiratory disease, or a combination thereof.

11. The method of claim 1, wherein the control level of the one or more markers is a level of the one or more markers in a sample obtained from a healthy subject.

12. The method of claim 1, wherein the level of the one or more markers or the control level of the one or more markers comprises an RNA level, a protein expression level or an activity of the one or more markers.

13. The method of claim 12, wherein the protein expression level of the one or more markers is determined using immunoassay or mass spectrometry.

14. The method of claim 13, wherein the immunoassay is an electrochemiluminescence assay, an enhanced chemiluminescence assay, an enzyme-linked immunosorbent assay (ELISA), or a lateral-flow assay (LFA).

15. The method of claim 13 wherein the mass spectrometry is matrix-assisted laser desorption/time of flight (MALDI/TOF) mass spectrometry, liquid chromatography quadruple ion trap electrospray (LCQ-MS), or surface-enhanced laser desorption ionization/time of flight (SELDI/TOF) mass spectrometry.

16. The method of claim 12, wherein the RNA level of the one or more markers is determined by RT-PCR.

17. The method of claim 2, wherein the therapeutic agent is selected from the group consisting of: isoniazid, rifampin, rifapentine, rifabutin, pyrazinamide, ethambutol, streptomycin, kanamycin, amikacin, moxifloxacin, gatifloxacin, levofloxacin, ofloxacin, ciprofloxacin aminocinomerizone, care, thiacetazone, clarithromycin, amoxicillin-clavulanic acid, imipenem, meropenem, clofazimine, viomycin, terizidone, TMS-207, PA-824, OPC-7683, LL-3858, SQ-109, and combinations thereof.

18. The method of claim 1, wherein the subject is a mammal.

19. The method of claim 1, wherein the subject is a human.

20. The method of claim 1, wherein the sample is a bodily fluid sample or a tissue sample.

21. The method of claim 1, wherein the sample is selected from the group consisting of blood, serum, and plasma.

22. The method of claim 1, wherein the sample is a blood sample.
